(19) United States
(12) Patent Application Publication
(10) Pub. No.: US 2011/0014131 A1
(43) Pub. Date: Jan. 20, 2011

(54) NANOMOLDING MICRON AND NANO SCALE FEATURES

(75) Inventors: Sandeep Disawal, Chicago, IL (US); Bjoem Rosner, Chicago, IL (US); Robert J. Janosky, Naples, FL (US)

Correspondence Address: FOLEY AND LARDNER LLP;SUITE 500, 3000 K STREET NW, WASHINGTON, DC, 20007, US

(73) Assignee: NanoInk, Inc.

(21) Appl. No.: 12/839,327

(22) Filed: Jul. 19, 2010

Related U.S. Application Data
(60) Provisional application No. 61/227,012, filed on Jul. 20, 2009.

(51) Int. Cl. A61K 9/48; A61J 3/06; B05D 5/00; B29C 45/14

(52) U.S. Cl. 424/10.2; 424/451; 424/456; 427/2.22; 264/265

(57) ABSTRACT

Small scale and nanoscopic identification features can be fabricated for pharmaceutical capsules. A composition comprising a capsule component, wherein the capsule component comprises a gelled pharmaceutical capsule material and at least one surface, wherein the at least one surface comprises at least one integral feature with a lateral dimension smaller than about 100 microns, or less than one micron. Methods of making the same are described by gelation with heating or cooling. The compositions and methods can be used for anti-counterfeiting.

### RELATED APPLICATIONS

This application claims priority to U.S. provisional Ser. No. 61/227,012 filed Jul. 20, 2009, which is hereby incorporated by reference in its entirety.

### BACKGROUND

A need exists to provide for better protection and security against counterfeiting and grey-market trading, particularly for pharmaceuticals. A review of counterfeiting in pharmaceuticals and its economic effects may be found in, for example, (1) “Counterfeit Pharmaceuticals: Current Status and Future Projections,” A. I. Wertheimer, et al. J. Am. Pharm. Assoc. 43(6) 710-718 (2003), and (2) Chapter 4 of the book Counterfeiting exposed: protecting your brand and your customers, D. M. Hopkins, L. T. Kontnik, M. T. Turnage (Wiley, Ed. 2003); ISBN: 0471269905. In addition, chapter 12 describes current anti-counterfeiting methods, such as holograms, intaglio printing, color-shifting technologies, and chemical or biochemical taggants. In many cases, however, prior methods are not appropriate or optimal for pharmaceuticals, since they would require the addition of a non-edible or unapproved chemical compound to the tablet or capsule.

### SUMMARY

Exemplary embodiments are summarized in this non-limiting summary section.

One embodiment comprises, for example, a composition comprising a capsule component, wherein said capsule component comprises a gelled pharmaceutical capsule material and at least one surface, wherein said at least one surface comprises at least one integral feature with a lateral dimension smaller than about 100 microns.

In some embodiments, such pharmaceutical capsule components comprise gelatin or methyl cellulose ethers, such as hydroxypropylmethyl cellulose. In some embodiments, such surfaces may be internal or external surfaces of the pharmaceutical capsule components. In some embodiments, such surfaces may be substantially concave or substantially convex. In some embodiments, such features may have a lateral dimension less than about one micron. In some embodiments, such features may have a height dimension less than about 100 microns or less than about 1 micron. In some embodiments such features may be bar codes or optically variable devices, including holograms.

Yet another embodiment comprises, for example, a method comprising: (i) providing at least one template comprising at least one first surface comprising at least one first feature with first lateral dimension smaller than about 100 microns; (ii) providing a pharmaceutical capsule material solution capable of gelation; (iii) coating said at least one first surface with said pharmaceutical capsule material solution; (iv) gelling said pharmaceutical capsule material solution to form at least one copy comprising at least one second surface with at least one second feature with second lateral dimension smaller than about 100 microns.

In some embodiments, such templates may comprise a receptacle and a removable insert, where the insert comprises the feature-bearing surface. In some embodiments, such templates may comprise molding pins or molds. In some embodiments, such surfaces of templates or copies may be substantially concave or substantially convex. In some embodiments, such features of template or copy surfaces may have a lateral dimension less than about one micron. In some embodiments, such features of template or copy surfaces may have a height dimension less than about 100 microns or less than about 1 micron. In some embodiments, such features of template or copy surfaces may be indentations or protrusions. In some embodiments, such features of template or copy surfaces may be bar codes or optically variable devices. In some embodiments, such coating may comprise such methods as dip coating, spray coating, spin coating, casting, extrusion blow molding, stretch blow molding, injection molding, and the like. In some embodiments, such gelation may comprise thermoreversible gel precipitation, polymer vitrification, polymer gelation, and the like. In some embodiments, such gelation may be reversible or irreversible.

Another embodiment provides a method comprising: (i) providing at least one template adapted for capsule formation comprising at least one first surface comprising at least one first feature with a first lateral dimension smaller than about one micron; (ii) providing a pharmaceutical capsule material solution capable of gelation; (iii) coating said at least one first surface with said pharmaceutical capsule material solution; and (iv) gelling said pharmaceutical capsule material solution to form at least one copy comprising at least one second surface with at least one second feature with a second lateral dimension smaller than about one micron.

Another embodiment provides a capsule component comprising gelatin and a surface, said surface comprising an integral feature with a lateral dimension smaller than about 100 microns.

Another embodiment provides a capsule component comprising gelatin and a surface, said surface comprising an integral feature with a lateral dimension smaller than about 1 microns.

Another embodiment provides a capsule component comprising hydroxypropylmethyl cellulose and a surface, said surface comprising an integral feature with a lateral dimension smaller than about 100 microns.

Another embodiment provides a capsule component comprising hydroxypropylmethyl cellulose and a surface, said surface comprising an integral feature with a lateral dimension smaller than about 1 microns.

Another embodiment provides a method comprising: (i) providing a template comprising a first surface comprising a first feature with a first lateral dimension smaller than about 100 microns; (ii) providing a gelatin solution capable of gelation; (iii) coating said at least one first surface with said gelatin solution; (iv) gelling said gelatin solution to form a copy comprising a second surface with a second feature with a second lateral dimension smaller than about 100 microns.

Another embodiment provides a method comprising: (i) providing a template comprising a first surface comprising a first feature with a first lateral dimension smaller than about one micron; (ii) providing a gelatin solution capable of gelation; (iii) coating said at least one first surface with said gelatin solution; (iv) gelling said gelatin solution to form a copy comprising a second surface with a second feature with a second lateral dimension smaller than about one micron.

Another embodiment provides a method comprising: (i) providing a template comprising a first surface comprising a first feature with first lateral dimension smaller than about 100 microns; (ii) providing an hydroxypropylmethyl cellulose solution capable of gelation; (iii) coating said at least one first surface with said hydroxypropylmethyl cellulose solution; (iv) gelling said hydroxypropylmethyl cellulose solution to form a copy comprising a second surface with a second feature with a second lateral dimension smaller than about 100 microns.

Another embodiment provides a method comprising: (i) providing a template comprising a first surface comprising a first feature with a first lateral dimension smaller than about one micron; (ii) providing an hydroxypropylmethyl cellulose solution capable of gelation; (iii) coating said at least one first surface with said hydroxypropylmethyl cellulose solution; (iv) gelling said hydroxypropylmethyl cellulose solution to form a copy comprising a second surface with a second feature with a second lateral dimension smaller than about one micron.

Another embodiment provides a method omprising: providing a template having a first surface, wherein the first surface has a first feature with a first lateral dimension smaller than about 100 microns; providing a pharmaceutical capsule material solution capable of gelation; coating said first surface with said pharmaceutical capsule material solution; and gelling said pharmaceutical capsule material solution to form a copy comprising a second surface with a second feature having a second lateral dimension smaller than about 100 microns; wherein said gelling is performed before said pharmaceutical capsule material solution is fully dried.

Another embodiment provides a pharmaceutical capsule having a substantially transparent shell, the capsule comprising: a structure inside the shell configured to form a diffraction pattern indicative an authenticity of the pharmaceutical capsule. In another embodiment, the shell is configured to couple light into and out the pharmaceutical capsule and to form a waveguide for the light. In another embodiment, the pharmaceutical capsule further comprises a metalized material inside the shell configured to reflect light to form the diffraction pattern.

Another embodiment provides a pharmaceutical capsule comprising: reflective particles configured to form coherent and controllably phase-shifted light upon illumination.

Another embodiment provides a method comprising: coupling light into a pharmaceutical capsule; detecting a diffraction pattern from reflected or transmitted light; and identifying the pharmaceutical capsule based on the diffraction pattern.

An advantage for at least one embodiment is that improved anti-counterfeiting can be achieved.

An advantage for at least one embodiment is that improved pharmaceutical compositions can be achieved without use of taggants or added chemicals.

### BRIEF DESCRIPTION OF FIGURES

The figures include scale bars and an approximate estimate of the showing of features is provided.

FIG. 1: Scanning electron microscopy of a gelatin film product of Example 1 showing features at, for example, a 100-500 micron scale.

FIG. 2: Scanning electron microscopy of a gelatin film product of Example 1 showing features at, for example, a 50-100 micron scale.

FIG. 3: Scanning electron microscopy of a gelatin film product of Example 1 showing features at, for example, a 10-50 micron scale.

FIG. 4: Scanning electron microscopy of a gelatin film product of Example 1 showing features at, for example, a 5-20 micron scale.

FIG. 5: Scanning electron microscopy of a gelatin film product of Example 1 showing features at, for example, a 1-10 micron scale.

FIG. 6: Scanning electron microscopy of a gelatin film product of Example 1 showing features at, for example, a 1-10 micron scale.

FIG. 7: Scanning electron microscopy of a gelatin film product of Example 1 showing features at, for example, a 1-10 micron scale.

FIG. 8: Scanning electron microscopy of a gelatin film product of Example 2 showing features at, for example, a 1 mm scale.

FIG. 9: Scanning electron microscopy of a gelatin film product of Example 2 showing features at, for example, a 100-500 micron scale.

FIG. 10: Scanning electron microscopy of a gelatin film product of Example 2 showing features at, for example, a 50-100 micron scale.

FIG. 11: Scanning electron microscopy of a gelatin film product of Example 2 showing features at, for example, a 20-50 micron scale.

FIG. 12: Scanning electron microscopy of a gelatin film product of Example 2 showing features at, for example, a 5-20 micron scale.

FIG. 13: Scanning electron microscopy of a gelatin film product of Example 2 showing features at, for example, a 5-20 micron scale.

FIG. 14: Scanning electron microscopy of a gelatin film product of Example 2 showing features at, for example, a 5-20 micron scale.

### DETAILED DESCRIPTION

All references cited hereinafter are incorporated by reference in their entirety. No admission is made that any of the cited references is prior art.

Priority U.S. provisional Ser. No. 61/227,012 filed Jul. 20, 2009 is hereby incorporated by reference in its entirety.

Embodiments described herein can be applied to a variety of compositions including pharmaceutical capsules, dietary supplements, and the like.

### Pharmaceutical Capsules

One embodiment is a pharmaceutical capsule, and pharmaceutical capsules are known in the art for a variety of uses. Pharmaceutical capsules may be designed to deliver an active pharmaceutical ingredient (API) to a specific portion of the body such as, for example, the gastrointestinal (GI) tract, where the API is then absorbed into the body. They should also be durable and stable during transport and storage. It is also desirable that they be palatable to the mouth and throat.

Alternatively, a pharmaceutical capsule may be designed to remain intact during its travel through the GI tract and exit with the stool. In this case, the pharmaceutical capsule may be used as, for example, a tracer in nutritional studies or API evaluation protocols.

Still other pharmaceutical capsules may be used as anal or vaginal suppositories. In these applications, it may be desirable that the capsules be easily inserted and not be irritating. Durability, stability, and effectiveness of API delivery will usually still be important considerations.

Capsules known in the art include, for example, hard gelatin, softgel, non-animal, and fish gelatin capsules. A variety of polymers can be used in the capsule including gelatin (including pharmaceutical grade gelatin), water-soluble polymers, and proteins. Capsules based on water-soluble polysaccharides can be used. Capsules based on, for example, pullulan and/or hypromellose can be used. Another embodiment for capsules is dietary supplements.

### Capsule Components

Pharmaceutical capsules can comprise capsule components. Capsule components can comprise, for example, a capsule body and a capsule cap. The capsule cap can be designed to fit tightly over the open end of the capsule body, to enclose the API. Some capsule bodies and capsule caps are designed with mechanical mechanisms to discourage tampering. Examples of such designs include U.S. Pat. Nos. 4,247,006 and 4,758,149, both of which are incorporated by reference in their entireties. These and other capsule component designs are well known to those skilled in the art.

### Capsule Materials

In some embodiments, capsule components comprise gelled pharmaceutical capsule materials. For capsules that are used in a human or animal, the materials should comply with applicable government regulations and not injure the host. For capsules that are intended to deliver an API to a specific part of the GI tract or to be used as suppositories, the materials should be selected to remain intact until the capsule is to dissolve. For capsules that are intended to remain intact during passage through the GI tract, the materials should be selected not to dissolve before exiting with the stool.

Examples of gelled pharmaceutical capsule materials include gelatin and methyl cellulose ethers, such as methyl cellulose and hydroxypropylmethyl cellulose. Suitable cellulosic materials are disclosed in, for example, U.S. Pat. No. 4,001,211, which is incorporated by reference in its entirety. These and other suitable materials are known to those skilled in the art.

### Capsule Material Solutions Capable of Gelation

In some embodiments, capsule components are manufactured from pharmaceutical capsule material solutions capable of gelation. Typical gelatin capsule component manufacturing processes are described in B. E. Jones, “Capsules, Hard,” entry in Encyclopedia of Pharmaceutical Technology, Vol. 2, pp. 251-258 (J. Swarbrick and J. C. Boylan, eds., Marcel Dekker, 1990), ISBN: 0824728017, which is incorporated by reference in its entirety. In such a process, gelatin is used as about, for example, a 25-30 weight percent aqueous solution at, for example, about 50-55° C. Pins at, for example, about 22° C. are dip coated in the solution. The gelatin solution on the pins subsequently gels as it cools and is dried. The resulting capsule components are then removed from the pins.

Similar processes have been disclosed for manufacturing capsule components from cellulose materials, such as methyl cellulose ethers, including, for example, hydroxypropylmethyl cellulose, in U.S. Pat. No. 5,698,155 and PCT publication WO 2008/050205, both of which are incorporated by reference in their entirety. These materials differ from gelatin in that they gel upon heating, rather than cooling. In these processes, pins are heated before being dip coated in cooler aqueous solutions of the methyl cellulose ethers. The pins continue to be heated after dip coating. The solution on the pins then gels and is dried. The resulting capsule components are then removed from the pins.

Generally, suitable capsule material solutions should be able to coat a surface, such as the surface of a molding pin, and undergo gelation to form capsule components. Gelation refers to physical or chemical transformations that allow the formed capsule components to retain the shape and surface features of the molding surface. Examples of such transformations include thermoreversible gel precipitation upon passing a sol through its transformation temperature, polymer vitrification upon cooling a melt or solution below its glass transition temperature, polymer gelation upon cross-linking or vulcanization beyond the gel point, and the like.

Depending on the intended application, it may be preferable that the gelation either be reversible or irreversible. For example, for the purpose of delivering an API to a specific portion of the GI tract, the gelation should be reversible under conditions seen in the body, so as to release the API at that location. On the other hand, if it is intended that the capsule remain intact until it leaves with the stool, the gelation should be irreversible under conditions seen in the body.

For systems amenable to thermoreversible gel precipitation, it may be appreciated that the sol-gel transformation temperature may generally be a function of the solution concentration. At the extremes, too dilute a solution may be incapable of gelation, while too concentrated a solution may gel too readily for practical use. At intermediate concentrations, the sol-gel transformation temperature will generally be a monotonic function of concentration. Suitable concentration ranges may be found by standard methods. For example, see N. Tanaka and I. Utsumi, “A Study of the Sol-Gel Transformation of Gelatin Solutions,” Bull. Chem. Soc. Japan, 36(1) 63-66 (1963), which is incorporated by reference in its entirety.

Although aqueous solutions are typical, solvents need not be limited to water. However, selection of solvents and process conditions should allow compliance with applicable workplace exposure, environmental, and pharmaceutical regulatory requirements.

### Templates, Coating, And Copies

In some embodiments, pharmaceutical capsule materials may be coated on surfaces of templates and allowed to gel. The type of template used will generally depend on the type of coating employed. The resulting gelled article may be referred to as a copy. In some embodiments, the copies are capsule components.

Molding pins are exemplary of templates used in dip coating. Molding pins suitable for use in a gelatin capsule dip coating process are disclosed in U.S. Pat. No. 4,758,149, which is incorporated by reference in its entirety. For other pharmaceutical compositions, because of differences in the extent of shrinkage upon gelation, the dimensions of the molding pins may differ from those used for gelatin compositions. An example of such pin modification for use with methyl cellulose ether compositions is disclosed in U.S. Pat. No. 5,756,036, which is incorporated by reference in its entirety.

Another example of a template is a mold, such as those used in such molding processes as casting, extrusion blow molding, stretch blow molding, injection molding, and the like. As in the case of molding pins, the design of molds must take into consideration shrinkage during gelation. Design of such molds is well known to those skilled in the art.

As will be clear to those skilled in the art, many other types of objects could be used as templates, when such using coating processes as spin coating or spray coating.

Template surfaces may be characterized as being substantially convex or substantially concave. An example of a substantially convex template surface is the outward-facing surface of a molding pin. On the other hand, an example of a substantially concave template surface is the inward-facing surface of a casting mold.

The surface of the template may be made of a variety of materials. In general, the surface should be chemically compatible with the gelled pharmaceutical capsule material solutions used in the method, so it does not corrode or contaminate the product. It is also desirable that the surface be harder and stiffer than both the gelled pharmaceutical capsule material solution and the gelled copy or capsule component. The surface material may optionally be treated to increase its hardness and durability, if desired, with diamond like coatings, nickel films, and the like. These considerations may be supplemented by others known to those skilled in the art.

Surfaces with Integral Features

In some embodiments, capsule components may have surfaces with integral features. “Integral features” refer to features consisting of the gelled capsule component material itself, rather than being other applied materials, such as inks or taggants. In will be appreciated by those skilled in the art that by avoiding the use of other materials, regulatory and fitness-for-use requirements may be more easily met. The integral feature can be characterized by an absence of an interface between the feature and the rest of the capsule.

Integral features may be either on an interior or exterior surface of capsule components.

Surfaces with Small Scale Features

In some embodiments, surfaces may have small scale features. Examples of such features are lines, dots, logos, bar-codes, and optically variable devices, including holograms. A variety of methods may be used to prepare such features. Examples of such methods are described in, for example, U.S. application Ser. Nos. 11/109,877 (filed Apr. 20, 2005), as well as 11/305,327; 11/305,189 and 11/305,326 (all filed Dec. 19, 2005) and these are hereby incorporated by reference in their entireties.

Optically variable devices are described in Lee, R.A., “Optically Variable Devices”, Chapter 7 of Micromanufacturing for Document Security, Mahalik, N. P. ed., Springer, Berlin, 2006, which is incorporated by reference in its entirety.

In some cases, lithographic methods may be used, including scanning probe lithography (including DPN printing, nanografting, nanooxidation, and scanning tunneling methods), electron beam lithography, ion beam lithography, laser-based lithography, optical lithography, ultraviolet lithography, X-ray lithography, electron projection lithography, ion projection lithography, low energy electron proximity lithography, forms of lithography involving neutral atoms, grey-tone (relief) microlithography, and the like. Lithographic methods may optionally be used in combination with other processing methods, for example, wet or dry etching, lift-off, substrate doping (including ion implementation), layer deposition, electroplating, electroless plating, polishing, chemical mechanical polishing, and the like. Alternatively, a suitable object may be provided by replicating the features of another object by, for example, stamping, or by molding into a soft material that is subsequently hardened and treated by physical vapor deposition, electroless plating, electroplating, or a combination thereof. These methods may be supplemented by others known to those skilled in the art.

For templates, the surface so prepared may either be an integral part of the template or, optionally, be part of a removable insert that fits into a receptacle in the template. Such a removable insert could enable the rapid changing of the surface used in the method, as might be required if the small scale features encoded such information as pharmaceutical lot numbers, product identifiers, manufacture dates, and the like.

Small scale features on the surface may be overt or covert. Overt features are those that are typically readily perceived by an observer without unusual technological assistance. Examples of overt features are described in U.S. application Ser. Nos. 11/109,877 filed Apr. 20, 2005, and 11/305,189 filed Dec. 19, 2005, both of which are incorporated by reference in their entireties. Such overt features may be used as means of authentication of objects or compositions of commercial value, such as pharmaceutical items. In such a case, it is preferable that the overt features be visually distinctive and of such a quality that they would be difficult for a counterfeiter to duplicate. Another possible use of such overt features would be to identify brands, models, pharmaceutical lot numbers, product identifiers, manufacture dates, doses, and the like.

Covert features are those that are typically difficult to detect, locate, or decode, especially with the naked eye or with conventional inspection technology, such as optical imaging. Examples of covert features are described in U.S. application Ser. Nos. 11/109,877 filed Apr. 20, 2005, and 11/305,326 filed Dec. 19, 2005, both of which are incorporated by reference in their entireties. Such covert features could enable detection of counterfeits without alerting counterfeiters of their presence. Alternatively, they might allow traceability of objects or compositions of commercial value, such as pharmaceutical items, by incorporating such information as pharmaceutical lot numbers, product identifiers, manufacture dates, and the like. Systems for detection of such covert features have been described in U.S. application Ser. No. 11/519,199 filed Sep. 12, 2006, which is incorporated by reference in its entirety.

Small scale features may be characterized by their dimensional measurements. One such dimensional measurement is the small scale feature's height, representing the feature's distance substantially above or below the surface. As used here, a feature's height is always a positive quantity, regardless of whether it lies above or below the surface. The height dimension is not particularly limited and can be, for example, about one micron or less, or more particularly, about 500 nm or less, or more particularly about 250 nm or less, or more particularly about 150 nm or less. There is no particular lower limit to the height dimension as long as the feature can be detected. The height dimension can be, for example, about one nm or more, or about 10 nm or more, or about 25 nm or more. Exemplary ranges can be, for example, about one nm to about one micron, or about 10 nm to about 500 nm, or about 25 nm to about 250 nm. If a pattern of repeating identification features is used, the height dimension can represent an average dimension.

Another such dimensional measurement is the small scale feature's lateral dimension, representing the feature's length or width substantially parallel to the surface. For a feature that is a line, the lateral dimension of length can be sufficiently long that the feature can be viewed with the naked eye or an optical microscope, whereas the lateral dimension of width can be sufficiently small that the feature cannot be so viewed. For applications requiring covert marks, one of these lateral dimensions can be made small. For example, the feature can have a lateral dimension of, for example, about 500 microns or less, or about 400 microns or less, or about 300 microns or less, or more particularly, about 250 microns or less, or more particularly, about 100 microns or less, or more particularly, about 10 microns or less. Or the feature can have a lateral dimension of, for example, about one micron or less, or more particularly, about 500 nm or less, or more particularly, about 250 nm or less, or more particularly, about 100 nm or less. There is no particular limit to how small the lateral dimension can be as long as the feature can be detected. For example, the lateral dimension can be at least about 1 nm, or more particularly, at least about 10 nm, or more particularly, at least about 100 nm, or more particularly at least about one micron. Hence, exemplary ranges for the lateral dimension include, for example, about one nm to about 500 microns, or about 10 nm to about 100 nm, or about 100 nm to about one micron, and about one micron to about 500 microns.

For barcodes, for example, the line length is not particularly limited but can vary from nanoscopic to microscopic. For example, lines can be about one micron to about 50 microns long, or about 5 microns to about 25 microns long, and yet have a line width of only about 50 nm to about 150 nm wide.

Where the small scale features are in the form of a pattern of repeating features, the features can be characterized by a lateral dimension representing an average lateral dimension such as an average circle diameter or an average line width.

Still another such dimensional measurement is the separation distance between small scale features or groups of small scale features. For example, if the features are a series of lines, a distance can be measured between the centers of the lines, or if the features are a series of dots, a distance can be measured between the centers of the dots. The distance of separation is not particularly limited, but smaller separation distances are preferred where it is desired that the features be covert. For example, one or more features can be separated from each other by an average distance of about 500 microns or less, or more particularly, about 100 microns or less, or more particularly, about 500 nm or less.

Yet still another such dimensional measurement is the size of the area of the smallest perimeter that could contain all of the members of a group of small scale features. This area can be, for example, about 10,000 square microns or less, or about 1,000 square microns or less, or about 400 square microns or less, or about 4 square microns or less, or about one square micron or less. Or the features can be, for example, in a square region with a lateral length and width of 100 microns×100 microns, or 20 microns×20 microns, or 2 microns×2 microns, or one micron×one micron. The groups need not, of course, be arranged in a square region. In general, the smallest perimeter might form a circle, a polygon, or some smooth or irregular closed shape.

As will be clear to those skilled in the art, it is possible for a surface to have many features that have a wide-range of dimensions. On can use one or more larger features in combination with one or more small scale features.

### Working Examples The following examples illustrate various embodiments of the claims. They are not intended to limit the scope of disclosure or claims.

### Example 1

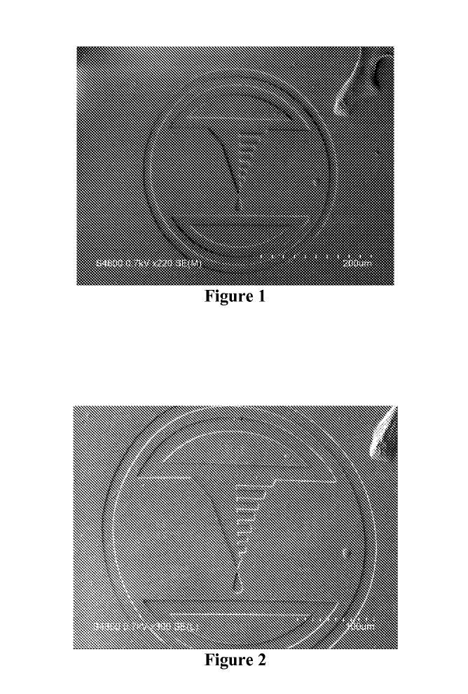

Gelatin capsules from Capsugel (Peapack, N.J.) were added to deionized water and heated to 90° C., allowing the gelatin to dissolve. A nickel strip bearing small scale features was dipped into the aqueous gelatin solution and removed. The film was allowed to cool and dry under ambient conditions. The gelled film was separated from the nickel strip. FIGS. 1-7 are scanning electron microscope images of the recovered gelatin film.

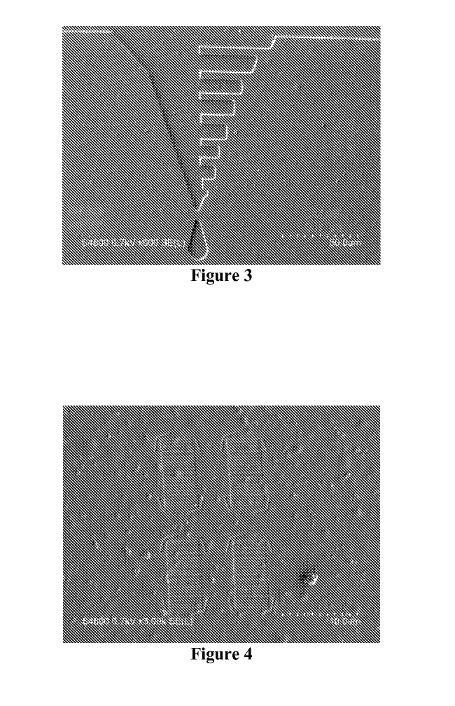

FIGS. 1-3 show gelatin film features on the micron scale. FIG. 1 shows a logo approximately 300 microns in diameter. FIG. 2 shows line widths of approximately 10-20 microns. FIG. 3 shows detailed features of approximately 10 micron scale.

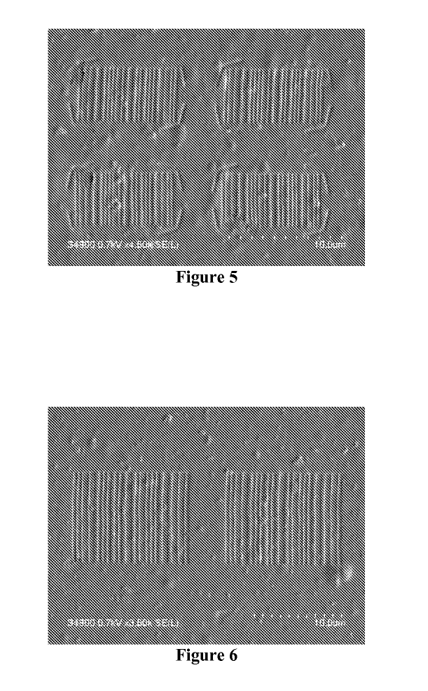

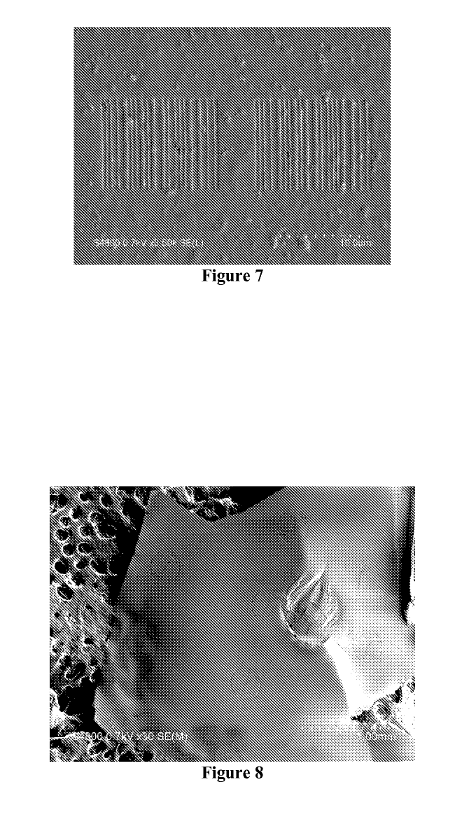

FIGS. 4-7 show gelatin film features on the sub-micron or nano scale. FIG. 4 shows four bar code regions, each approximately 100-150 micrometers in length and approximately 40-60 micrometers in width. FIG. 5 is a closer view of the same bar code regions. FIGS. 6 and 7 are views of two bar code regions, where each bar is approximately 0.3-0.6 microns in width.

### Example 2

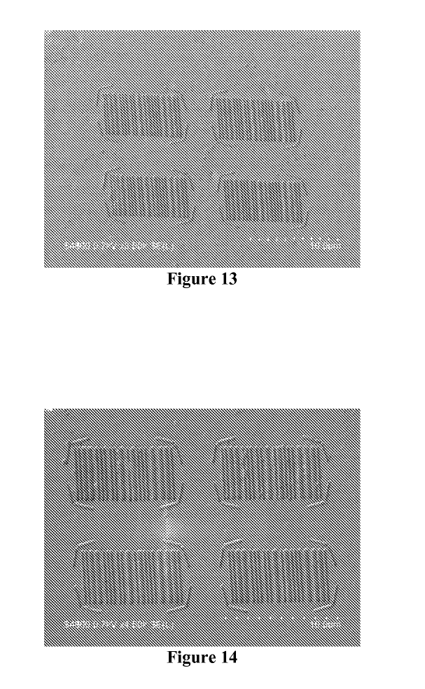

A capsule pin was fitted with a receptacle on its free end. An insert with a surface bearing small scale features was inserted into the receptacle. Gelatin capsules from Capsugel were added to deionized water and heated to 90° C., allowing the gelatin to dissolve. The pin with insert was dipped into the aqueous gelatin solution, so that the insert and the adjacent part of the pin were coated. The pin with insert was subsequently removed from the solution. The film was allowed to cool and dry under ambient conditions. The gelled film was separated from the pin with insert. FIGS. 8-13 are scanning electron microscope images recovered gelatin film.

FIG. 8 shows several micron-scale logos on the gelatin film. Each is separated from its nearest neighbor by approximately 1 mm.

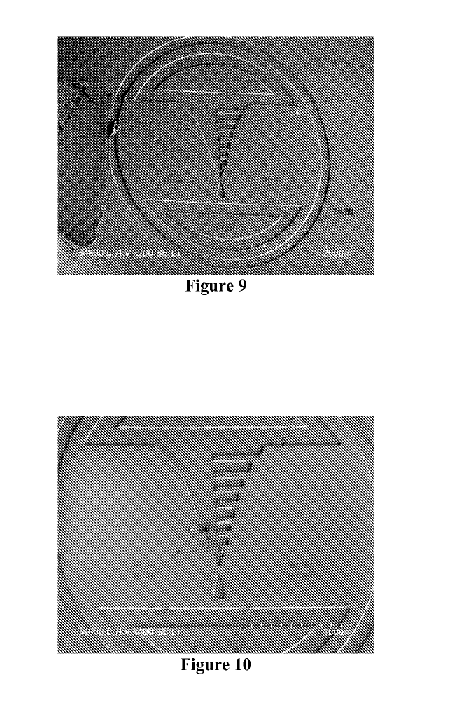

FIGS. 9 and 10 show two different features at the micron level. FIG. 9 shows a logo approximately 300 microns in diameter. FIG. 10 shows line widths of approximately 10-20 microns.

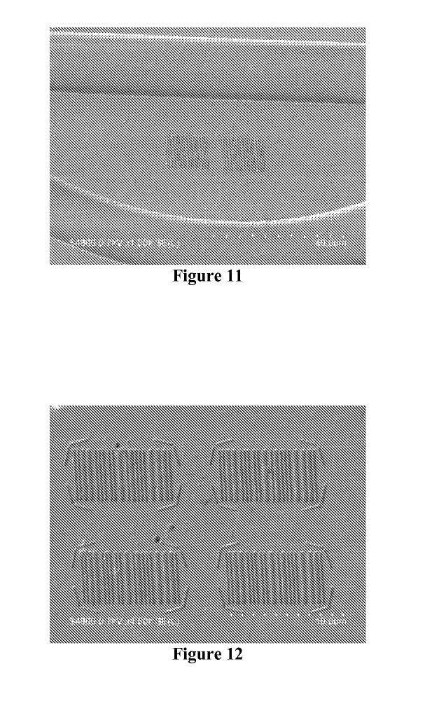

FIG. 11 shows two barcodes within one of the micron-scale logos. They are separated from each other by approximately 10 microns.

FIGS. 12-14 show gelatin film features on the sub-micron or nano scale. All three show four bar code regions, each approximately 100-150 micrometers in length and approximately 40-60 micrometers in width.

### Nanoencryption on Partially Dried or Undried Gelatin

Imprinting methods with stamps are described in, for example, U.S. patent application Ser. Nos. 11/109,877 filed Apr. 20, 2005; 11/305,327 filed Dec. 19, 2005; 11/305,189 filed Dec. 19, 2005; and 11/305,326 filed Dec. 19, 2005. See also U.S. Patent publication 2010/0046825. In some additional embodiments, such imprinting can be made on partially dried or undried material to be gelled such as, for example, gelatin or HPMC. After the pins dip into the gelatin they can be in a process of being dried for extended periods. Contact with the stamp (on the exterior of the shell) can be made before the material is fully dried and hardened.

This process may require no additional heat for molding. Drying action can help with demolding, similar to how in nanoimprint lithography cooling helps by going below Tg. This however is a different process. Stamps could be built in one large sheet to comply with dipping pin form factor. Imprinting could happen by bringing a stamp head assembly into contact when the capsules are stationary or in motion on the drying belt. Another way to do stamping could be dice or stamp mounted on a roller and making contact with capsules as they pass by. Stamps could stay in contact with a given set of pins for the entire drying process. Feature depth and remaining capsule thickness at thinnest point can be controlled mechanically. Other advantages may include a much faster throughput and no requirement of additional machinery, and thus lower cost. Stamps with only logos can be used to extend stamp life and to offer a low cost solution (if required by users).

### Nano-Molding for Transparent Capsules

Nano-molding can be performed on a capsule interior for transparent capsules, which can have a visible inner surface. This can be very useful for anti-counterfeiting purposes, and can be complementary to some other methods that may not work with transparent capsules. In one embodiment, only overt (logo) features are used that will be seen with a basic authentication kit. They are protected from environmental effects. They can be suitable for liquid filled capsules.

Detection can be made using a standard reflected light, or light transmission if the capsule is either fully transparent or disassembled.

A type of dark-field illumination can be used. Light can be coupled into the capsule shell like in a waveguide. When the light hits the sharp edges of the integral features formed from the imprinting, the light will be scattered and become visible, providing an optical signature.

Coupling the light into the capsule shell can be through, for example, refraction-index-matched gels and glues, and a glass window. Alternatively, the capsule can be submersed into a suitable liquid inside a transparent tank.

With this type of illumination it may be possible to obtain diffraction patterns without surface metallization, provided that the light is properly coupled into and out of the capsule shell.

Holograms can be obtained when viewing in transmission (e.g., illuminating from inside the dome) due to the phase change imparted by the different optical path length imparted in the surface relief. The inside of a capsule can be metalized to obtain diffraction patterns when illuminating from the outside.

What is claimed:

1. A method comprising:
(i) providing at least one template comprising at least one first surface comprising at least one first feature with a first lateral dimension smaller than about 100 microns;
(ii) providing a pharmaceutical capsule material solution capable of gelation;
(iii) coating said at least one first surface with said pharmaceutical capsule material solution; and
(iv) gelling said pharmaceutical capsule material solution to form at least one copy comprising at least one second surface with at least one second feature with a second lateral dimension smaller than about 100 microns.

2. The method according to claim 1, wherein said at least one template comprises at least one receptacle and at least one removable insert, wherein said at least one receptacle is adapted to accept said at least one removable insert, wherein said at least one removable insert is adapted to fit in said at least one receptacle, and wherein said at least one insert comprises said at least one first surface.

3. The method according to claim 1, wherein said at least one template comprises at least one molding pin.

4. The method according to claim 1, wherein said at least one template comprises at least one mold.

5. The method according to claim 1, wherein said at least one first surface is substantially convex.

6. The method according to claim 1, wherein said at least one first surface is substantially concave.

7. The method according to claim 1, wherein said at least one first feature has a first height dimension smaller than about 100 microns.

8. The method according to claim 1, wherein said at least one first feature has a first height dimension smaller than about one micron.

9. The method according to claim 1, wherein said first lateral dimension is smaller than about one micron.

10. The method according to claim 1, wherein said at least one first feature comprises at least one indentation into said at least one first surface.

11. The method according to claim 1, wherein said at least one first feature comprises at least one protrusion out of said at least one first surface.

12. The method according to claim 1, wherein said at least one first feature comprises at least one bar code.

13. The method according to claim 1, wherein said at least one first feature comprises at least one hologram.

14. The method according to claim 1, wherein said pharmaceutical capsule material solution comprises gelatin.

15. The method according to claim 1, wherein said pharmaceutical capsule material solution comprises at least one methyl cellulose ether.

16. The method according to claim 1, wherein said pharmaceutical capsule material solution comprises hydroxypropylmethyl cellulose.

17. The method according to claim 1, wherein said coating comprises dip coating.

18. The method according to claim 1, wherein said coating comprises molding.

19. The method according to claim 1, wherein said coating comprises spray coating.

20. The method according to claim 1, wherein said at least one copy comprises at least one capsule cap.

21. The method according to claim 1, wherein said at least one copy comprises at least one capsule body.

22. The method according to claim 1, wherein said at least one second surface is substantially convex.

23. The method according to claim 1, wherein said at least one second surface is substantially concave.

24. The method according to claim 1, wherein said at least one second feature has a second height dimension smaller than about 100 microns.

25. The method according to claim 1, wherein said at least one second feature has a second height dimension smaller than about one micron.

26. The method according to claim 1, wherein said second lateral dimension is smaller than about one micron.

27. The method according to claim 1, wherein said at least one second feature comprises at least one indentation into said at least one second surface.

28. The method according to claim 1, wherein said at least one second feature comprises at least one protrusion out of said at least one second surface.

29. The method according to claim 1, wherein said at least one second feature comprises at least one bar code.

30. The method according to claim 1, wherein said at least one second feature comprises at least one hologram.

31. A composition comprising a capsule component, wherein said capsule component comprises a gelled pharmaceutical capsule material and at least one surface, wherein said at least one surface comprises at least one integral feature with a lateral dimension smaller than about 100 microns.

32. The composition according to claim 31, wherein said capsule component is a capsule cap.

33. The capsule component according to claim 31, wherein said capsule component is a capsule body.

34. The composition according to claim 31, wherein said gelled pharmaceutical capsule material comprises gelatin.

35. The composition according to claim 31, wherein said gelled pharmaceutical capsule material comprises at least one methyl cellulose ether.

36. The composition according to claim 31, wherein said gelled pharmaceutical capsule material comprises hydroxypropylmethyl cellulose.

37. The composition according to claim 31, wherein said at least one surface is substantially convex.

38. The composition according to claim 31, wherein said at least one surface is the exterior of a capsule cap.

39. The composition according to claim 31, wherein said at least one surface is the exterior of a capsule body.

40. The composition according to claim 31, wherein said at least one surface is substantially concave.

41. The composition according to claim 31, wherein said at least one surface is the interior of a capsule cap.

42. The composition according to claim 31, wherein said at least one surface is the interior of a capsule body.

43. The composition according to claim 31, wherein said at least one integral feature has a height dimension smaller than about 100 microns.

44. The composition according to claim 31, wherein said at least one integral feature has a height dimension smaller than about one micron.

45. The composition according to claim 31, wherein said lateral dimension is smaller than about one micron.

46. The composition according to claim 31, wherein said at least one integral feature comprises at least one indentation into said at least one surface.

47. The composition according to claim 31, wherein said at least one integral feature comprises at least one protrusion out of said at least one surface.

48. The composition according to claim 31, wherein said at least one integral feature comprises at least one bar code.

49. The composition according to claim 31, wherein said at least one integral feature comprises at least one optically variable device.

50. The composition according to claim 31, wherein said at least one integral feature comprises at least one hologram.

51. A method comprising:
(i) providing at least one template adapted for capsule formation comprising at least one first surface comprising at least one first feature with a first lateral dimension smaller than about one micron;
(ii) providing a pharmaceutical capsule material solution capable of gelation;
(iii) coating said at least one first surface with said pharmaceutical capsule material solution; and
(iv) gelling said pharmaceutical capsule material solution to form at least one copy comprising at least one second surface with at least one second feature with a second lateral dimension smaller than about one micron.

52. The method of claim 51, wherein gelling is carried out by heating the capsule material.
